(19) United States
(12) Patent Application Publication
(10) Pub. No.: US 2009/0041698 A1
(43) Pub. Date: Feb. 12, 2009

(54) Cosmetic product in the form of compact powder comprising a message informing the user

(75) Inventors: Lily-Ann Cabiling, Beaugency (FR); Sylvain Maimon, Saint Jean Le Blanc (FR); Eric Perrier, Les Cotes D'arey (FR)

Correspondence Address: HAMRE, SCHUMANN, MUELLER & LARSON, P.C., P.O. BOX 2902, MINNEAPOLIS, MN, 55402-0902, US

(73) Assignee: LVMH RECHERCHE, Saint Jean De Braye (FR)

(21) Appl. No.: 11/977,516

(22) Filed: Oct. 25, 2007

(30) Foreign Application Priority Data
Aug. 10, 2007 (FR) 0757044

(51) Int. Cl. A61K 8/00; A45D 33/18; A61Q 1/00

(52) U.S. Cl. 424/69; 132/293

(57) ABSTRACT

A compressed powder product (60) intended for cosmetic use comprises at least one string of alphanumeric characters (24) displayed essentially throughout said usable thickness h and forming a message that is intended to inform the user of said product about at least one commercial, technical or regulatory characteristic of said product, said characters themselves consisting of a cosmetically acceptable substance, preferably also a compact powder, and being visually distinct from the rest of said product so that said message remains legible on the surface of said product during use, thereby implementing a method of informing the user about the cosmetic product.

The present invention relates to a product in the form of compact powder intended for cosmetic use, and to a cosmetic composition in the form of compact powder or incorporating a compact powder as an essential constituent.

It further relates to a process for the preparation of this composition and to a device for carrying out this process.

It further relates to a method of informing the user of the cosmetic product about certain characteristics of this product.

It is conventional to diffuse cosmetic products into packagings and to display a number of items of information about the nature or composition of the product, or any other characteristic of a regulatory or commercial type, on the receptacle in which the product is sold, on the box containing this receptacle or on a data sheet contained inside this box.

The present invention results from the idea of its inventors to display such information not on a part outside the product (packaging or data sheet) but directly within the product, and in such a way that this message remains legible to the user throughout the use of the product.

This invention offers a real advantage for the user, who has permanent access to useful information throughout the use of the product, e.g. even when the data sheet is lost, which is very common.

It is already known, especially for purely decorative purposes, to display an ornamental element on the surface of a solid cosmetic product, especially a compact powder.

An example which may be cited is Spanish patent ES 20027585, which describes the insertion of a decorative element on the surface of a powder or within a fatty substance such as a lipstick.

However, it will be noted that said document simply describes the case where the decorative element is inserted within the fatty substance of the lipstick type, or on the surface of a compressed powder, without attempting to insert the decorative motif throughout the usable thickness of the product.

International patent application WO 2007/072149 describes a cosmetic product into which non-cosmetic decorative elements are introduced in well-defined places.

European patent application EP 0432808 describes a process for the preparation of a cosmetic product with powders of different colors or different characteristics, wherein a succession of layers of these different powders is produced and compressed and an excavation step is then carried out to define a visible surface formed of adjacent portions of these different layers.

The object of this process is to provide the user with compact powders of different colors within one and the same container.

Processes are also known which make it possible to create a decorative motif on the surface of a cosmetic composition. International patent application WO 03/072069, wherein an image is created by laser irradiation on the surface of the compressed powder, may be cited in particular.

French patent application FR 2 759 941, which describes a process for decorating the surface of a cosmetic powder using an inkjet printer, may also be cited.

Thus, although the prior art has already described cosmetic compositions carrying a decorative motif, either on the surface or inside the composition, no attempt has ever been made to display, within the composition itself, a message that is intended to inform the consumer, let alone a message that remains apparent and legible throughout the use of the composition.

Thus, according to one of its essential characteristics, the present invention relates to a product in the form of compact powder, or essentially consisting of compact powder, which is intended for cosmetic use, said product comprising at least one string of alphanumeric characters displayed essentially throughout its usable thickness and forming a message that is intended to inform the user of said product about at least one commercial, technical or regulatory characteristic of said product, said characters themselves consisting of a cosmetically acceptable substance, preferably also a compact powder, and being visually distinct from the rest of said product so that said message remains legible on the surface of said product during use.

Products in the form of compact powder are well known in the field of cosmetics and more particularly in the field of make-up.

Within the framework of the invention, “product essentially consisting of powder” defines a product consisting of at least 60% by weight of powder, the remainder consisting of a liquid mixture composed e.g. of liquids, a pasty or semi-pasty liquid solution or waxes, comprising fatty substances, silicones or perfumes, and more particularly of liquids serving as binders to form the above-mentioned cohesive, compact product.

Moreover, “essentially throughout the thickness” further defines the fact that the string of alphanumeric characters is produced throughout the thickness or essentially throughout the thickness, the latter case signifying that the alphanumeric characters can stop very close to the lower or upper surface of the cohesive product, e.g. about 0.1 to 3 mm from said surface, particularly about 0.1 to 1 mm from said surface.

Within the framework of the invention, the compact powder can be obtained by any technical manufacturing process well known to those skilled in the art.

In a first embodiment, there may be mentioned the preparation of this compact powder by compressing a mixture of dry, cosmetically acceptable constituents, themselves pulverulent, comprising at least 60% by weight of dry powder, and a liquid mixture composed e.g. of a liquid, pasty or semi-pasty liquids or waxes, comprising fatty substances, silicones or perfumes, and more particularly liquids serving as binders to form the above-mentioned compact product.

In a second embodiment, there may be mentioned the preparation of this compact powder by casting or molding a mixture of cosmetically acceptable constituents in the form of a powder wetted with a cosmetically acceptable solvent such as water, or a pasty or semi-pasty mass, and then drying the latter to give the above-mentioned compact powder, especially as described in WO 05/084617 or WO 04/05708.

The above-mentioned cosmetic product, in the form of compact powder or essentially consisting of compact powder, can comprise one or more constituents selected from an excipient, preservatives, pigments, pearlescent agents, compressive or cohesive agents, binders, preferably fatty binders, texturizing agents, sun protection agents or filters, etc. All these constituents are well known to those skilled in the art.

The preservatives can be present in an amount of approximately or exactly 1% by weight of the composition.

The pigments can be present in a proportion of between 0 and 30% by weight of the composition.

The pearlescent agents can be present in a proportion of between 0 and 80% by weight of the composition, preferably of between 0 and 50% by weight of the composition and particularly preferably of between 1 and 50% by weight of the composition.

The compressive or cohesive agents can be present in a proportion of between 0 and 10% by weight of the composition, preferably of between 2 and 5% by weight of the composition. In the absence of pigment, the shade of the compressed powder is that of the mixture of the constituents of its formulation, i.e. generally a very light or white shade.

The fatty binders can be present in a proportion of between 0 and 30% by weight of the composition, preferably of between 5 and 14% by weight of the composition.

One or more perfumes are also used in a proportion generally of between 0 and 2% by weight of the composition, preferably of 0.1 to 1% by weight of the composition.

The texturizing agents can be present in a proportion generally of between 0 and 50% by weight of the composition, preferably of between 1 and 20% by weight of the composition.

The sun protection agents or filters for protecting the skin from the effects of the sun, particularly the UV A and UV B, can be present in a proportion that is necessary to provide the desired protection from the sun, this proportion generally being between 0 and 30% of the final composition.

Excipients generally make the composition up to 100%, particularly excipients that enable additional characteristics to be imparted to the product.

The above-mentioned cosmetic product, in the form of compact powder or essentially consisting of compact powder, will be especially an eye shadow, a blush-type product, a product for illuminating the face, a make-up foundation, a sun powder or any other powder-based cosmetic product of galenical form.

As explained previously, what makes the present invention original and useful is the fact that the product of the invention offers a long-lasting display of information useful to the consumer, this information being displayed permanently on the surface of the compact powder during the use of the product, remaining permanently in the structure of the product itself.

It will be very evident to the reader that, during the use of the cosmetic product in the form of compact powder or essentially consisting of compact powder, the usable thickness of the product decreases because the user removes product.

Now, by virtue of the invention, the string of characters forming a message intended for the user can remain visible on the surface of the product, said message being inscribed throughout the usable thickness of the cosmetic product.

The alphanumeric characters forming the message themselves consist of a cosmetically acceptable substance, which will preferably also be a compact powder.

The alphanumeric characters will advantageously consist of at least one compact powder whose shade and/or degree of compression and/or texture and/or composition will render the message visually distinct so as to make it legible on the surface of the cosmetic product at any time during use.

Of course, the shade of the compact powder constituting the characters will have to be chosen according to the desired visual effect and also so as not to interfere with the desired final effect of the cosmetic composition.

According to another characteristic of the invention, the make-up effect of the product in powder form removed from the whole of its surface must be satisfactory.

In addition, according to another characteristic of the invention, the powder product must have a satisfactory level of disintegration or crumbling resistance at the moment of removal and, when applied to the face, a shade that the consumer considers compatible with use as a cosmetic.

All these characteristics are obtained by the powder cohesion techniques well known to those skilled in the art, e.g. compression of the powder in a manner similar to the compression of the commercially available compressed powders, or by the molding or casting of a powder wetted with a cosmetically acceptable solvent such as water, followed by drying and, if appropriate, by compression as well.

This compression, or molding and drying, can be effected manually or by automatic means in a manner well known to those skilled in the art.

For essentially esthetic reasons, the characters forming the message consist at least in part of dashes, lines and curves whose width l, measurable on the surface of the product, is less than 7 mm, preferably less than 5 mm and particularly preferably less than 3 mm throughout the use of the product.

The information that is permanently visible on the surface of the product during use is advantageously information pertaining to the commercial, technical or regulatory characteristics of the product.

Examples of such information which may be mentioned are the name of the product, its mark, the various characteristics of the product, the shade, the sun protection factor (SPF), the batch number of the product, its expiration date or the letters SLAO (shelf life after opening), QD (quasi-drug) or OTC (over-the-counter), the origin of the product and specifically the country in which it was manufactured (Made in . . . ), or any other information, particularly of a regulatory nature, that is useful to the consumer.

Thus the invention makes it possible to display on the powder itself, throughout its use, information that is useful to the consumer, especially regulatory information, the cosmetic product itself therefore serving as an information carrier.

This information is generally displayed in the form of a text made up of alphanumeric characters, i.e. letters and/or numbers. It is specified in this regard that, without exceeding the framework of the present invention, said alphanumeric characters in the string forming the message can be preceded, followed or separated by one or more non-alphanumeric symbols (such as periods, hyphens, commas, asterisks, etc.) or by spaces.

For the sake of convenience, in the remainder of the present text the expression “alphanumeric message” is understood as meaning a message as defined above, formed of a string of alphanumeric characters.

In one preferred mode of carrying out the invention, the alphanumeric message consists of a string of at least three alphanumeric characters.

The invention further relates to the process for the preparation of the cosmetic products of the invention.

In a first embodiment, this process comprises at least one step in which at least two cosmetic powders are compressed or compacted, one of said powders being intended to form at least one alphanumeric character under conditions that effect its insertion throughout or essentially throughout the usable thickness of said product.

In one particular mode of the invention, this process comprises:
providing at least one mold comprising a compression cavity which is provided with at least one partition whose shape, width and height are adapted to the formation, in its interior space, of each alphanumeric character for which provision is made within the cosmetic product, the number of partitions corresponding to the desired number of alphanumeric characters;
filling the mold cavity outside the partitions with a first cosmetic powder composition;
filling the interior of the partitions with a second cosmetic powder composition intended to form the desired alphanumeric message, it furthermore being equally possible for this filling step to be carried out before the filling of the exterior space;
carrying out a step in which the cosmetic powder outside the partitions and the cosmetic powder inside the partitions are compressed, preferably simultaneously, to give a precompressed powder, which those skilled in the art generally refer to as cake; and
demolding the resulting precompressed cosmetic powder or cake.

In one preferred mode of carrying out the process described above, a set of screens is used at the moment of filling with the powders, the shape of said screens being adapted to the space which it is desired to blank off, and the purpose of said screens being alternately to obstruct the space outside the partitions and then the space forming the alphanumeric message, according to the order in which the spaces are filled with the powder compositions, so as to prevent part of a mixture intended for filling one of the spaces from accidentally contaminating the other space, thereby e.g. degrading the colors or technical characteristics of the final product.

In the case where it is desired to use the same mold to finalize the shape of the compressed powder product, movable partitions are used; these are removed after precompaction and the powder is compressed again, particularly in a manner similar to the commercially available compressed powders, so as to eliminate the spaces left by the partitions in order to give a finished product in the form of compressed powder of substantially uniform surface.

In the case where fixed partitions are used, the demolded precompressed powder or cake is transferred to another mold which does not contain such partitions but in which a small packaging cup is preferably inserted, so as to carry out said recompression in order to give a finished product in the form of compressed powder of substantially uniform surface in its packaging cup.

In one or other of the variants (movable or fixed partitions), the precompressed powder is preferably placed in a cup before the final compression, said cup forming the packaging in which the finished product is placed.

In the case where movable partitions are used, the cup is preferably placed in the mold before filling with the powder, whereas in the case where a mold with fixed partitions is used, the cakes are transferred after precompression to a mold which accommodates inside it the cup constituting the primary packaging of the compressed powder.

In one particularly preferred mode of carrying out the invention, the filling volume of the powders in each of the compartments is adapted so that, once the final compression has been carried out, the height of compact powder is equal or very substantially equal to the height of the walls of the commercial packaging cup, whereby the compact powder is flush with the top of the cup partitions and has an esthetic appearance.

After the final compression of the powder, the cup containing the compressed powder is expelled from the mold, either by turning the mold upside-down or by ejecting the cup, in order to be packaged in a secondary packaging, e.g. a case, a box or a device of the powder compact type, optionally including a device for removing the powder and, if desired, a mirror.

In the case where the shade of the first composition and the shade of the second composition are different, a product in the form of compact powder is obtained which comprises within it a message formed of alphanumeric characters that is visually distinct from the rest of the product, so that this message remains legible on the surface of the product during use. According to one variant, the surface of the product can be non-smooth so as to present the alphanumeric message above or below the surface.

The process used according to the invention thus amounts, in a way, to the manufacture of a compact powder having several shades, in which the information has a different shade from the bulk of the product in which it is inserted, so that the information can be read. In another variant, it is also possible to make provision for the alphanumeric message to have several different shades in order to give the alphanumeric message a pleasant or “modern” visual effect.

This process makes it possible to ensure that the message which it is desired to display appears sharp and precise and is displayed throughout the usable thickness of the compressed powder.

Those skilled in the art will obviously understand that this process is derived from that used to make powders having several shades, but requires the manufacture of molds adapted to the particular shape of the characters forming the message which it is desired to print.

According to another characteristic, the invention relates to a device for carrying out the above process.

In one embodiment, this device comprises:
at least a first set of molds comprising a first mold having a cavity in which partitions are inserted whose shape is adapted to define the desired alphanumeric message, and whose height H corresponds to the height H of said cavity (12), and a countermold or die containing grooves corresponding to the thickness of the partitions, and
if the partitions are movable, a second countermold or die not containing grooves but having a continuous compression surface,
or if the partitions are fixed, at least a second set of molds not containing partitions, which is intended to accommodate the product obtained after the first compression step and, preferably placed in a commercial packaging cup, is subjected to a second compression step by means of a countermold or die not containing grooves but having a continuous compression surface.

Compression with each countermold or die can be effected manually or by automated compression means.

As indicated previously, the final compression value to obtain the finished product does not need to be different from the final compression value for the commercially available cosmetic products in the form of compact powder, e.g. compressed powder.

Further, the invention relates to a method of informing a user of a cosmetic product of a characteristic of the cosmetic product, the method comprising:

providing a cosmetic product comprising a compact powder having a useable thickness, and a cosmetically acceptable substance provided in the form of a alphanumeric message visible in the usable thickness of said cosmetic product;

communicating to the user at least one non-visible characteristic of said product through the user reading the alphanumeric message, wherein the user is informed of the non-visible characteristic of the cosmetic product.

In a particular embodiment, the non-visible characteristic is a commercial, technical or regulatory characteristic.

Finally, according to a last characteristic, the invention relates to a method of informing the user of a cosmetic product in the form of compact powder or incorporating a compact powder as an essential constituent, wherein said product is provided with at least one string of alphanumeric characters displayed essentially throughout the usable thickness of said product and forming a message for informing the user of said product about at least one commercial, technical or regulatory characteristic of said product, said characters themselves consisting of a cosmetically acceptable substance, preferably also a compact powder, and being visually distinct from the rest of said product, so that said message remains legible on the surface of said product during use.

Other objects, characteristics and advantages of the invention will become clearly apparent from the following explanatory description referring to the attached Figures, which comprise a currently preferred embodiment of a manufacturing device according to the present invention, as well as several Examples, which are given simply by way of illustration and cannot therefore in any way limit the scope of the invention.

In the Figures:

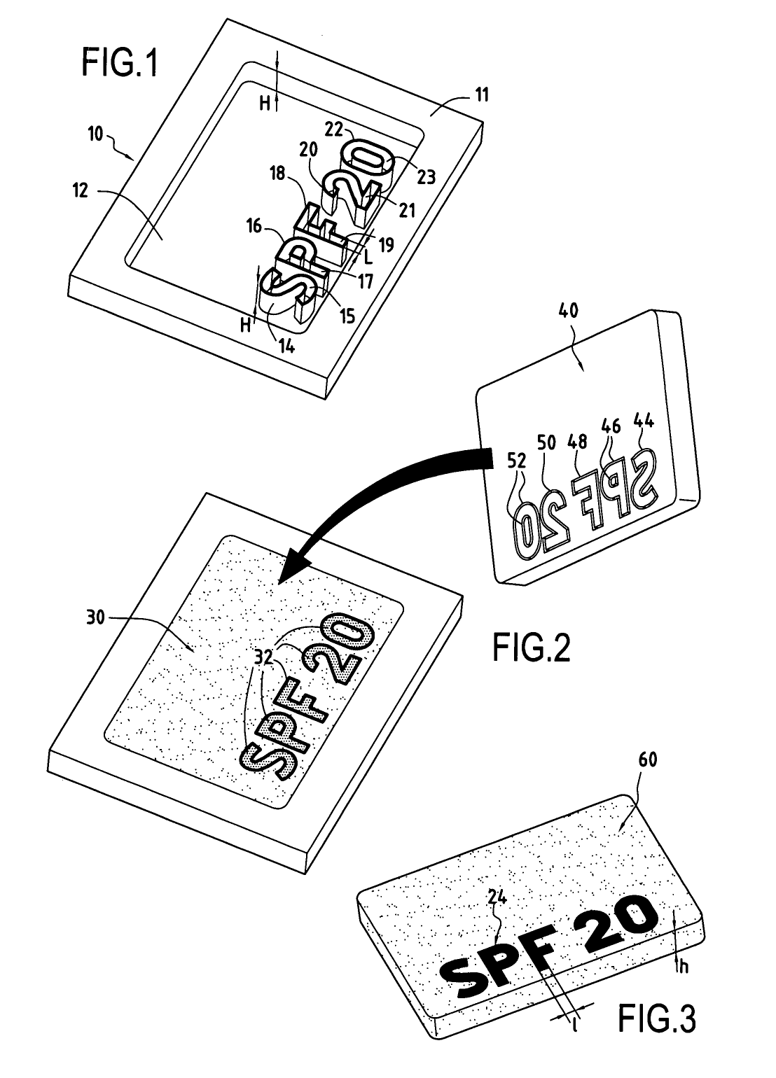

FIG. 1 shows a mold which comprises a cavity for containing the powder to be compressed, and into which a commercial packaging cup for the compact powder can be inserted, it being possible for said mold to be provided with several partitions whose shape is appropriate for the desired string of alphanumeric characters, whose height H can advantageously be substantially equal to the height of the mold cavity, which itself corresponds to the filling level of the powder before the precompression or compression step, H being very much greater than the usable height or thickness h of the powder product in the form of compressed powder according to the invention (h being equal or very similar to the height of the wall of the commercial cup in which the compact powder is packaged), and whose width is intended to create the desired width l of these alphanumeric characters;

FIG. 2 shows the mold of FIG. 1 into which a first cosmetic powder composition has been poured outside the partitions and a second cosmetic powder composition has been poured inside the partitions, said second composition being shown here with a different shade, and the second part of the mold, namely a countermold forming a die, which comprises grooves whose shape and depth correspond to the shape, thickness and height of the partitions intended for forming the desired alphanumeric message of desired character width l, the arrow symbolically indicating the step in which the countermold or die is applied against the surface of the powder in order to compress it; after this first compression, if they are of movable type, the partitions adapted for forming the alphanumeric message can advantageously be removed, after which a second compression is effected with another countermold or die not containing grooves, so as to carry out the final compression of the product, preferably in its commercial packaging cup, in a manner well known to those skilled in the art;

FIG. 3 shows the ready-to-use product according to the invention in the form of compressed powder, but in this case without its packaging cup in order to facilitate understanding of the final product.

With reference to FIGS. 1 to 3, it is seen that a device according to the invention, represented by the general reference number 10, for the manufacture of a product in the form of compact powder, compressed in this example, or a product incorporating a compact powder, compressed in this case, as an essential constituent, and intended for cosmetic use, comprises a first mold 11 having a cavity 12 whose shape is adapted to the shape of the finished compressed product and whose height H corresponds to the filling height of the mold that is necessary to fill the cavity with the uncompressed powder, in order to obtain a finished product of height h corresponding to the usable thickness of the finished product 60, as shown in FIG. 3.

Within the framework of the invention, this cavity 12 is advantageously provided with partitions 14, 16, 18, 20, 22 whose shape is adapted to correspond to the alphanumeric characters, identified by the general reference number 24, that form the desired alphanumeric message in the finished product. These partitions have a height H identical or similar to the height H of the cavity 12, and an internal width L defining an interior space 15, 17, 19, 21, 23 respectively, whose dimensions correspond to the width l of the desired alphanumeric message in the finished product, as can easily be understood by those skilled in the art.

This device according to the invention also comprises at least one countermold or die 40, shown in FIG. 2, which comprises, in its bulk, grooves 44, 46, 48, 50, 52 respectively, whose shape corresponds to the shape and thickness of the partitions 14, 16, 18, 20, 22 respectively, and whose internal width and depth also correspond to the height H of said partitions, so that the countermold or die can be applied against a powder that is to be poured into the cavity 12 and the spaces 15, 17, 19, 21, 23, as explained below, without being impeded by the partitions themselves, as can easily be understood by those skilled in the art.

Thus, referring to FIG. 2, it is seen that, after the cavity 12 has been filled with a first powder composition to be compressed, represented by the general reference number 30, said first composition being cosmetically acceptable and being capable of corresponding to a conventional cosmetic powder composition, the spaces 15, 17, 19, 21, 23 inside the partitions 14, 16, 18, 20, 22 are also filled with a second cosmetic powder composition, represented by the general reference number 32, which, in the example shown, has a different color from that of the powder composition poured into the cavity 12 outside the partitions, as shown.

Using the countermold or die 40, a first compression step, or precompression step, is then carried out on the two powders 30 and 32, in this case simultaneously.

The precompressed powder, or cake, in which the presence of the grooves corresponding to the partitions previously existing in the mold 11 will be noted, can then be demolded.

Preferably, this cake will be placed in a commercial packaging cup and the cup/cake assembly will be placed in the cavity of the final compression mold to subsequently facilitate the packaging of the final compact product.

In a first variant, the partitions 15, 17, 19, 21, 23 may be removed if they are of movable type relative to the mold 11, and at this moment, preferably after the precompressed product or cake has been placed in a packaging cup, the precompressed powder product comprising the composition 30 and the composition 32 may be subjected to a final compression, making it possible to eliminate the grooves formed in the powder by the previous presence of the partitions, to give a finally compressed, finished product 60 having a height h, no longer containing grooves, and a width l of the alphanumeric characters, as shown in FIG. 3, preferably in its commercial packaging cup, which is not shown here in order to facilitate understanding of the finished product.

In a second variant, provision can be made for the precompressed product, containing the mark of grooves corresponding to the partitions 15, 17, 19, 21, 23, preferably after having been placed in a commercial packaging cup, to be placed, preferably in its cup, in a second mold having a cavity not comprising partitions, so as to be able to carry out a final compression with a countermold or die also devoid of grooves, i.e. having an entirely continuous compression surface, in order to give the final product as shown in FIG. 3.

The surface of the countermold or die can be non-planar so as to define reliefs or recesses over all or part of the surface of the cohesive product, thereby creating specific visual or esthetic effects.

Several Examples of the composition of powders used to prepare several products in the form of compressed powder according to the invention are given below; once again, they are given simply by way of illustration and cannot in any way limit the scope of the invention.

### EXAMPLES OF THE INVENTION

### Example 1

### Preparation of a Compressed Powder P1 According to the Invention

1.1 A first cosmetic powder mixture having the following composition (% expressed by weight) is prepared:

| | |
|---|---|
| Preservatives | 0.6% |
| Dehydroacetic acid sodium salt | |
| Tetrasodium EDTA | |
| Cohesive agents | 3.5% |
| Magnesium stearate | |
| Magnesium myristate | |
| Physical filter | 10% |
| (UV-TITANE M 160) | |
| Texturizing agents | 49% |
| PMMA (MICROMA ® 100) | |
| Polyamide 12 (ORGASOL 2002 EX | |
| D NAT COS) | |
| Cellulose | |
| Treated sericite | |
| Treated mica | |
| Binder | 15.85% |
| (Silicone DOW CORNING 9041 and | |
| FLUID DC | |
| 556 and dimethicone/PEG crosspolymer | |
| (KSG-210)) | |
| Perfume | 0.1% of the final composition |
| Chemical filters (octyl methoxycinnamate | 8% of the final composition |
| and 3-benzophenone) | |
| Pigments (iron and titanium oxides) | 5.86% |
| Excipient (talc, mica) | qsp 100% |

This first mixture is intended to form the surroundings of the message “SPF 20”.

The colors of the iron oxides used as pigments are yellow, red and black.

Different shades are mixed to give a shade 1 light beige color (very low intensity).

1.2 A second cosmetic powder mixture having the following composition (percentage expressed by weight) is prepared:

| | |
|---|---|
| Preservatives | 0.6% |
| Dehydroacetic acid sodium salt | |
| Tetrasodium EDTA | |
| Cohesive agents | 3.5% |
| Magnesium stearate | |
| Magnesium myristate | |
| Physical filter | 10% |
| (UV-TITANE M 160) | |
| Texturizing agents | 49% |
| PMMA (MICROMA ® 100) | |
| Polyamide 12 (ORGASOL 2002 | |
| EX D NAT COS) | |
| Cellulose | |
| Treated sericite | |
| Treated mica | |
| Binder | 15.85% |
| (Silicone DOW CORNING 9041 and | |
| FLUID DC 556) | |
| Dimethicone/PEG crosspolymer | |
| (KSG-210) | |
| Perfume | 0.1% of the final composition |
| Chemical filters (octyl methoxycinnamate | 8% of the final composition |
| and 3-benzophenone) | |
| Pigments (iron oxides) | 8.5% |
| Excipient (talc, mica) | qsp 100% |

The second mixture has a shade 4 beige/brown color (intense) that differs from that of the first mixture, the intensity of which is adapted so that the final shade, resulting from the mixing of the two shades at the moment of use of the product, is of intermediate intensity (shade 2 or 3).

1.3 Compression of these Mixtures with the Device Shown in FIGS. 1 to 3

The compressed powder P1 according to the invention is prepared by filling the cavity 12 outside the partitions 14, 16, 18, 20, 22 with the first mixture defined in section 1.1 above, and the spaces 15, 17, 19, 21, 23 inside the partitions 14, 16, 18, 20, 22 respectively, with the second mixture defined in section 1.2 above.

These two powders are then subjected to a first compression or pre-compression by means of the countermold or die 40 comprising the grooves 44, 46, 48, 50, 52.

The precompressed powder or cake is then demolded.

This precompressed powder comprising both the first and second mixtures can then be placed in another mold devoid of internal partition(s), which is compressed to the desired usable height h or thickness with another countermold or die not containing the grooves 44, 46, 48, 50, 52, i.e. having a planar and continuous compression surface, thereby producing the desired compressed powder 60 according to the invention, called P1, shown in FIG. 3.

The final product is a “sun powder”, i.e. a tanning powder with sun protection, which is applied to the cheeks, forehead, nose and chin and provides a tanning effect while at the same time protecting the skin from the effects of exposure to the sun.

It is seen that the compressed powder obtained comprises an alphanumeric message of the desired width l as indicated previously, in this case “SPF 20”, which here indicates the value of the sun protection factor which will be visible to the user throughout the life of the product.

### Example 2

### Compressed Powder P2 According to the Invention

2.1 A first cosmetic powder mixture having the following cosmetic composition (percentage expressed by weight) is prepared:

| Talc | qsp |
|---|---|
| Preservatives | 1.5% |
| Compressive or cohesive agents (magnesium stearate) | 0.5% |
| Binder (pentaerythrityl tetraisostearate) | 12% |
| Texturizing agents (polyamide powder) | 7.1% |
| Pearlescent agent (silicone-treated sericite) | 50% |
| Pigments (yellow, black, red iron oxide) | 2% |

This first mixture is intended to form the alphanumeric characters of the words “MADE IN FRANCE” and will produce a caramel shade.

2.2 A second powder mixture is prepared which has the same cosmetic composition as the first powder mixture above, except for the absence of pigments, which are replaced by talc in this case. The shade of the powder is a lighter beige.

This mixture is intended to form the surroundings of the words “MADE IN FRANCE”.

2.3 Preparation of the Compressed Powder

The preparation is the same as that described for the compressed powder P1 according to the invention, but obviously using partitions of appropriate shapes to form the alphanumeric message MADE IN FRANCE.

This gives a compressed powder P2 according to the invention, in which the color of the alphanumeric message, in this case MADE IN FRANCE, is caramel, whereas the color of the surroundings of this message is beige.

The product obtained is a face illuminator, i.e. a make-up product for illuminating the complexion of the face.

The invention includes all the means that constitute technical equivalents of the means described and shown in the attached Figures.

What is claimed is:

1. A product in the form of compact powder, or essentially consisting of compact powder, which is intended for cosmetic use, said product comprising at least one string of alphanumeric characters displayed essentially throughout said usable thickness (h) and forming a message that is intended to inform the user of said product about at least one commercial, technical or regulatory characteristic of said product, said characters themselves consisting of a cosmetically acceptable substance, and being visually distinct from the rest of said product so that said message remains legible on the surface of said product during use.

2. The product of claim 1, wherein said string of alphanumeric characters forming said message consists at least in part of dashes, lines or curves whose width (l), measurable on the surface, is less than 7 mm, preferably less than 5 mm and particularly preferably less than 3 mm throughout the use of the product.

3. The product of claim 1, wherein said string of alphanumeric characters consists of letters and/or numbers forming a message intended to give the user information about the name or the characteristics of said product, preferably information of a commercial or regulatory nature.

4. The product of claim 1, wherein said message consists of a string of at least three alphanumeric characters.

5. The product of claim 1, wherein said message is the name of the product, its shade, its sun protection factor (SPF), its batch number, its expiration date (SLAO) or the place of manufacture “Made in . . . ”.

6. The product of claim 1, wherein said string of alphanumeric characters consists of at least one compact powder whose color and/or degree of cohesion and/or relief makes said message visually distinct.

7. A process for the preparation of a product as defined in claim 1, which comprises at least one step in which at least two cosmetic powders are compressed or compacted, one of said powders being intended to form at least one alphanumeric character under conditions that effect its insertion throughout or essentially throughout the usable thickness of said product.

8. The process of claim 7, which comprises:
a) providing at least one mold comprising a compression cavity which is provided with at least one partition whose shape, width and height are adapted to the formation, in its interior space, of each alphanumeric character for which provision is made within the cosmetic product, the number of partitions corresponding to the desired number of alphanumeric characters;
b) filling the mold cavity outside the partitions with a first cosmetic powder composition;
c) filling the interior of the partitions with a second cosmetic powder composition intended to form the desired alphanumeric message, it furthermore being equally possible for this filling step to be carried out before the filling of the exterior space;
d) carrying out a step in which the cosmetic powder outside the partitions and the cosmetic powder inside the partitions are compressed to give a precompressed powder or cake;
e) demolding the resulting precompressed cosmetic powder or cake; and
i. either, when using the same mold to finalize the shape of the compressed powder product, providing movable partitions; said movable partitions being removed at this stage and the powder or cake is compressed again, to eliminate the spaces left by the partitions in order to give a finished product in the form of compressed powder of substantially uniform surface; or
ii. when using fixed partitions, the demolded precompressed powder or cake is transferred to another mold which does not contain such partitions, so as to carry out said recompression in order to give a finished product in the form of compressed powder of substantially uniform surface.

9. The method of claim 8 wherein the transfer is performed after the precompressed powder or cake has been placed in a commercial packaging cup.

10. A device for carrying out the process according to claim 7, which comprises:
at least a first set of molds comprising a first mold having a cavity in which partitions are inserted whose shape is adapted to define the desired alphanumeric message, and whose height H corresponds to the height H of said cavity, and a countermold or die containing grooves corresponding to the thickness of the partitions, and
if the partitions are movable, a second countermold or die not containing grooves but having a continuous compression surface,
or if the partitions are fixed, at least a second set of molds not containing partitions, which is intended to accommodate the product obtained after the first compression step and, preferably placed in a commercial packaging cup, is subjected to a second compression step by means of a countermold or die not containing grooves but having a continuous compression surface.

11. A method of informing a user of a cosmetic product of a characteristic of the cosmetic product, the method comprising:
providing a cosmetic product comprising a compact powder having a useable thickness, and a cosmetically acceptable substance provided in the form of a alphanumeric message visible in the usable thickness of said cosmetic product;
communicating to the user at least one non-visible characteristic of said product through the user reading the alphanumeric message, wherein the user is informed of the non-visible characteristic of the cosmetic product.

12. The method of claim 11, wherein the non-visible characteristic is a commercial, technical or regulatory characteristic.
